(19) United States
(12) Patent Application Publication
(10) Pub. No.: US 2024/0346213 A1
(43) Pub. Date: Oct. 17, 2024

(54) FEEDBACK-BASED PREDICTION OF MEDICAL DEVICE DEFECTS

(71) Applicants: Fresenius Medical Care Deutschland GmbH, Bad Homburg (DE); Fresenius Medical Care AG, Bad Homburg (DE)

(72) Inventors: Jannik Müller, Darmstadt (DE); Norman Förster, Marburg (DE); Bernd Lunghamer, Frankfurt (DE); Radomir Popovic, Berlin (DE)

(21) Appl. No.: 18/582,267

(22) Filed: Feb. 20, 2024

(30) Foreign Application Priority Data
Apr. 17, 2023 (EP) 23168257.6

(51) Int. Cl. G06F 30/27; G16H 40/40

(52) U.S. Cl. CPC G06F 30/27; G16H 40/40

(57) ABSTRACT

A computer-implemented method for training a model for predicting medical device defects includes: predicting, by the model, a future fault condition of a medical device based on a current operating condition of the medical device; determining a complexity of the predicted fault condition; transmitting the predicted fault condition to a receiver based at least in part on the complexity of the predicted fault condition; receiving information about an actual operating condition of the medical device; and adjusting a set of parameters of the model based at least in part on the information about the actual operating condition and the predicted fault condition of the medical device. The medical device may be, for example, a hemodialysis (HD) device or a peritoneal dialysis (PD) device.

### CROSS-REFERENCE TO RELATED APPLICATION

Priority is claimed to European Patent Application No. EP 23168257.6, filed on Apr. 17, 2023, the entire disclosure of which is hereby incorporated by reference herein.

### FIELD

The present disclosure relates to a computer-implemented method for generating feedback for a model for predicting medical device defects. In addition, a corresponding model for predicting medical device defects, a method for training this model, as well as a corresponding data processing apparatus and a computer program, are part of the present disclosure.

### BACKGROUND

Medical devices nowadays have a multiplicity of sensors which enable continuous monitoring of operating parameters. An important aspect here is the ensuring of functional and safe medical devices in everyday clinical practice. On account of device faults, caused e.g. by component wear, a plurality of functional medical devices usually have to be kept available within the scope of good risk management, with the result that the care of patients is nevertheless ensured in the event of a fault.

On the one hand, fewer patients can thus be cared for at the same time than would be possible with a given number of devices. On the other hand, withholding additional devices, e.g. two devices, is insufficient under unfavorable circumstances: in rare cases, for example, unexpected faults occur in more than two devices in one day, with the result that treatments that are necessary have to be postponed.

Once the fault has occurred, fault states can prove to be entirely complex. As a rule, a simple visual check or analysis of the operating parameters is then not sufficient to obtain a well-founded knowledge of the cause of the fault. The cause finding is then the responsibility of the commissioned service technician, who has to bring along a corresponding wealth of experience and expert knowledge for this purpose.

### SUMMARY

In an exemplary embodiment, the present disclosure provides a computer-implemented method for training a model for predicting medical device defects. The method includes: predicting, by a computing system implementing the model, a future fault condition of a medical device based on a current operating condition of the medical device; determining, by the computing system, a complexity of the predicted fault condition; transmitting, by the computing system, the predicted fault condition to a receiver based at least in part on the complexity of the predicted fault condition; receiving, by the computing system, information about an actual operating condition of the medical device; and adjusting, by the computing system, a set of parameters of the model based at least in part on the information about the actual operating condition and the predicted fault condition of the medical device.

In another exemplary embodiment, the present disclosure provides a non-transitory computer-readable medium having processor-executable instructions stored thereon for training a model for predicting medical device defects. The processor-executable instructions, when executed, facilitate performance of the following: predicting, by a computing system implementing the model, a future fault condition of a medical device based on a current operating condition of the medical device; determining, by the computing system, a complexity of the predicted fault condition; transmitting, by the computing system, the predicted fault condition to a receiver based at least in part on the complexity of the predicted fault condition; receiving, by the computing system, information about an actual operating condition of the medical device; and adjusting, by the computing system, a set of parameters of the model based at least in part on the information about the actual operating condition and the predicted fault condition of the medical device.

In yet another exemplary embodiment, the present disclosure provides a computing system for training a model for predicting medical device defects. The computing system includes: one or more memories having processor-executable instructions stored thereon; and one or more processors configured to execute the processor-executable instructions to facilitate the following being performed: predicting, by the model, a future fault condition of a medical device based on a current operating condition of the medical device; determining a complexity of the predicted fault condition; transmitting the predicted fault condition to a receiver based at least in part on the complexity of the predicted fault condition; receiving information about an actual operating condition of the medical device; and adjusting a set of parameters of the model based at least in part on the information about the actual operating condition and the predicted fault condition of the medical device.

### BRIEF DESCRIPTION OF THE DRAWINGS

Exemplary embodiments of the present application will be described in even greater detail below based on the exemplary figures. The application is not limited to the exemplary embodiments. All features described and/or illustrated herein can be used alone or combined in different combinations in embodiments of the application. Features and advantages of various embodiments of the present application will become apparent by reading the following detailed description with reference to the attached drawings which illustrate the following:

FIG. 1 shows an overview of a system for predicting medical device defects according to aspects of the present disclosure.

FIG. 2 shows a prediction of a medical device defect including receiver feedback according to aspects of the present disclosure.

FIG. 3 shows a section of an exemplary training file for training a model for predicting medical device defects according to aspects of the present disclosure.

### DETAILED DESCRIPTION

Exemplary embodiments of the present disclosure provide an efficient method for predicting medical device defects in order to ensure the availability and safety of medical devices and thus reduce the occurrence of actual fault cases via timely maintenance or repair.

A first aspect relates to a computer-implemented method for training a model for predicting medical device defects. The method can comprise: predicting, by the model, a future fault condition of a medical device based on a current operating condition of the medical device; determining a complexity of the predicted fault condition; transmitting the predicted fault condition to a receiver based at least in part on the complexity of the predicted fault condition; receiving information about an actual operating condition of the medical device; and adjusting a set of parameters of the model based at least in part on the information about the actual operating condition and the predicted operating condition of the medical device.

More efficient training of the model is achieved by transmitting the predicted fault condition to a receiver based on the complexity of the predicted fault condition. This is due to the fact that the corresponding receiver is able to evaluate the prediction of the model correctly, thereby creating robust evidence for adjusting the parameters of the model.

According to a further aspect, adjusting the set of parameters comprises: determining a difference between the actual operating condition and the predicted operating condition, wherein adjusting the set of parameters is further based on the determined difference.

By determining the difference, it can be determined in which points the prediction does not correspond to the actual operating condition. A targeted adjustment of the parameters can thus take place.

According to a further aspect, determining the difference between the actual operating condition and the predicted operating condition is based on a created feedback file, wherein a structure of the created feedback file is based at least in part on the complexity of the predicted fault condition and/or on the capability profile of the receiver, and wherein the structure of the feedback file specifies a level of detail of the adjusted feedback file.

The feedback file enables a feedback loop which takes into account not only the complexity of the fault condition but also the capabilities of the receiver. Depending on these factors, a level of detail of the feedback file can be selected which enables the best possible determination of the difference.

According to a further aspect, the structure of the feedback file comprises one or more binary questions regarding the predicted fault condition of the medical device, one or more predefined condition descriptions of the medical device and/or one or more action instructions associated with the predicted fault condition.

By a combination of binary questions, predefined condition descriptions and associated action instructions, a detailed matching between predicted fault condition and actual operating condition can be carried out.

According to a further aspect, the future fault condition of the medical device comprises: a prediction of a probability of failure of the medical device within a predefined future time period, one or more predictions of one or more probabilities of failure of one or more individual components of the medical device and/or one or more predictions of one or more probabilities of cause of failure of the one or more individual components.

The gradations between the individual probabilities enables a detailed cause control or evaluation.

According to a further aspect, the prediction of the future fault condition of the medical device comprises: making, by a first sub-model of the model, the prediction of the probability of failure of the medical device within the predefined future time period, making, by a first plurality of sub-models of the model, the one or more predictions of the one or more probabilities of failure of the one or more individual components of the medical device and/or making, by a second plurality of sub-models of the model, the one or more predictions of the one or more probabilities of cause of the failure of the one or more individual components.

The assembly of the model via a plurality of sub-models enables a harmonization of the prediction of the individual probabilities.

According to a further aspect, adjusting the set of parameters of the model comprises: determining, according to the actual operating condition of the first sub-model of the model, a sub-model of the first plurality of sub-models of the model and/or a sub-model of the second plurality of sub-models of the model; and adjusting a set of parameters of the determined at least one sub-model.

Through the targeted determination of the sub-model which is responsible for an incorrect prediction, unnecessary training of the other sub-models can be prevented. Instead, it is sufficient to adjust only the set of parameters of the determined sub-model.

According to a further aspect, the method further comprises: pre-training the model using a plurality of training data as input for the model, wherein a training file of the plurality of training data comprises: a fault condition of the medical device that has occurred at a past time, a fault log about an operating condition of the medical device before the past time, and a repair log including actions to eliminate the fault condition that has occurred.

In order to avoid excessive load on the system, the model can already be pre-trained on a corresponding training data set. The model is thus already able to correctly estimate many operating conditions. Adjusting the set of parameters is therefore only performed in specific cases (e.g. those cases that have not yet been comprised by the training data).

According to a further aspect, the transmitting comprises: determining a capability profile of the receiver and matching the determined complexity with the capability profile of the receiver, wherein the capability profile comprises at least: a capability of the receiver to evaluate a fault condition up to a predefined complexity.

By matching the complexity with the capability profile of the receiver, it is avoided that the fault condition to be evaluated is incorrectly transmitted to a receiver which does not have the necessary capabilities.

A second aspect relates to a computer-implemented method for predicting medical device defects, comprising: predicting, by a model, a future fault condition of a medical device based on a current operating condition of the medical device, wherein the model has been trained according to a method according to one of the aspects disclosed above.

According to a further aspect, the current operating condition of the medical device comprises: usage data of the medical device, technical device data of the medical device and/or environmental data of the medical device, wherein the future fault condition of the medical device comprises: a fault diagnosis, a fault probability and/or a time period.

A third aspect relates to a model for predicting medical device defects, wherein the model has been trained according to a method of an aspect disclosed above.

According to a further aspect, the model comprises: a first sub-model of the model, for predicting a probability of failure of the medical device within a predefined future time period; a first plurality of sub-models of the model, for predicting one or more probabilities of failure of one or more individual components of the medical device; and/or a second plurality of sub-models of the model, for predicting one or more probabilities of cause of the one or more individual components.

A fourth aspect relates to a data processing apparatus configured for carrying out a method according to any one of the aspects disclosed herein and/or for storing and using the model according to any one of the aspects disclosed herein.

A fifth aspect relates to a computer program comprising instructions which, when executed by a computer, cause the computer to carry out a method according to any one of the aspects disclosed above.

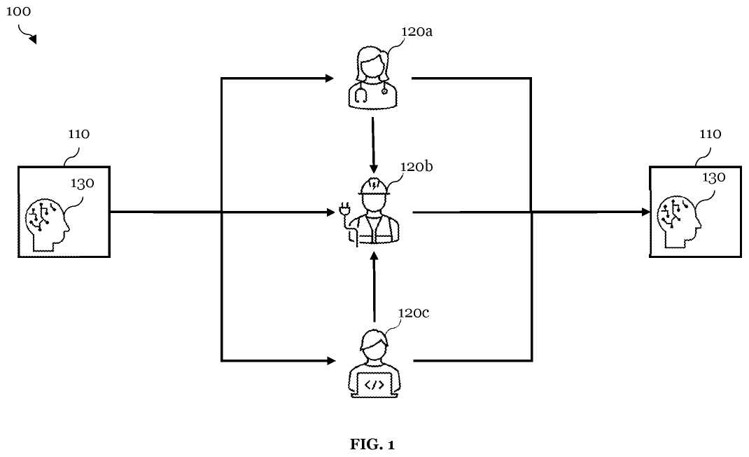

FIG. 1 shows an overview of a system 100 for predicting medical device defects according to aspects of the present disclosure. Within this system 100, a method for training a model for predicting medical device defects according to aspects of the present disclosure can also take place. The system 100 can comprise a medical device 110 (e.g. a dialysis device such as hemodialysis (HD) devices or peritoneal dialysis (PD) devices, etc.). The medical device 110 can comprise a multiplicity of sensors for monitoring an operating condition of the medical device 110. The medical device 110 can further comprise corresponding components and functionality (e.g. processor, memory, transceiver for corresponding communication technologies such as Message Queuing Telemetry Transport (MQTTS)) for collecting, processing, sending and receiving data (such as the sensor data). The system can further comprise a model 130 for predicting medical device defects such as the medical device 110. The model 130 can be both part of the medical device 110 and part of a server (e.g. cloud server). The medical device can e.g. include a data processing apparatus comprising components for storing and using the model 130. In this case, the sensor data collected by the sensors of the medical device 110 are forwarded directly to the data processing apparatus in order to make a corresponding prediction. However, it is also possible that the model 130 is hosted on a data processing apparatus such as a server. In this case, the sensor data of the medical device 110 are first transmitted to the data processing apparatus via corresponding communication technology (e.g. MQTTS, Secure File Transfer Protocol (SFTP), Hypertext Transfer Protocol Secure (HTTPS), etc.) in order to subsequently make a corresponding prediction. The illustration in FIG. 1 is therefore only used for clarification. In any case, based on a current operating condition of the medical device 110, a prediction can be made by the model 130 about a future fault condition of the medical device 110. In addition, the prediction can also be based on one or more historical operating conditions.

The system 110 can additionally comprise one or more receivers 120a-c. The predicted (future) fault condition can be transmitted to one or more of the receivers 120a-120c. A receiver can subsequently acquire information about an actual operating condition of the medical device 110 (e.g. via an evaluation of the predicted fault condition based on the current operating condition of the medical device 110). Should the predicted fault condition correspond to the actual operating condition of the medical device 110, this can be confirmed by the corresponding receiver. In this case, adjusting a set of parameters of the model 130 (i.e. re-training the model 130) would not be necessary. Should, however, the predicted fault condition not correspond to the actual operating condition of the medical device 110 (i.e. there is a difference between the actual operating condition as indicated via the information about the actual operating condition of the medical device and the predicted fault condition of the medical device 110), the set of parameters of the model 130 can be adjusted based at least in part on the information about the actual operating condition and the predicted fault condition. The communication between the individual entities of the system 100 can take place via corresponding interface communication. For this purpose, for example, a Representational State Transfer (REST) application programming interface (API) can be implemented as an interface via which, for example, the predicted fault condition can be transmitted to one or more of the receivers 120a-c. On the other hand, via a corresponding interface call, the information about the actual operating condition can in turn be transmitted from the corresponding receiver 120a-c to the model 130, thereby enabling any adjustment of the set of parameters of the model.

The receivers 120a-c can be, for example, a nurse 120a, a service technician 120b and remote maintenance personnel 120c. In this case, each receiver 120a-c can be associated with a corresponding capability profile. This can comprise, for example, a capability of the receiver to evaluate a fault condition up to a predefined complexity. Accordingly, the transmission of the predicted (future) fault condition of the medical device 110 to one or more receivers 120a-c can depend on a complexity of the predicted fault condition. A capability profile of the nurse 120a can comprise, for example, a capability to evaluate a fault condition of low complexity. A capability profile of the remote maintenance personnel 120c can comprise, for example, a capability to evaluate a fault condition of medium complexity. A capability profile of the service technician 120b can comprise, for example, a capability to evaluate a fault condition of high complexity.

A fault condition can have a low complexity if the fault condition is, for example, visually verifiable (e.g., whether a bibag connector is correctly attached or has a crack). This fault condition could then be transmitted to each of the receivers 120a-c, since each receiver has, according to its capability profile, the capability to evaluate a fault condition of (at least) low complexity.

A fault condition can have a medium complexity if the fault condition is, for example, not visually verifiable (e.g., if it requires reading out a device fault memory of the medical device 110). This fault condition could then only be transmitted to the receivers 120b-c, since these have, according to their capability profiles, the capability to evaluate a fault condition of medium complexity, while the receiver 120a can only evaluate fault conditions of low complexity and thus does not come into consideration.

A fault condition can have a high complexity if the fault condition requires, for example, disassembly of the medical device. This fault condition could then only be transmitted to the receiver 120b, since only this has, according to its capability profile, the capability to evaluate a fault condition of high complexity, while the receivers 120a-b can only evaluate fault conditions of low or medium complexity. In the case that a fault condition of too high a complexity has nevertheless been transmitted to the corresponding receiver for the latter, this can be forwarded to the appropriate receiver (i.e. the receiver which can evaluate the complexity). This can have taken place, for example, on the basis of an incorrect assessment of the model 130 (for example if the model 130 has not yet been sufficiently trained). The determination of the complexity of a fault condition can take place, for example, via a look-up table which contains a corresponding complexity for each fault condition. However, the determination of the complexity can also take place on the basis of an additional output of the model 130.

Whether a difference between the actual operating condition and the predicted fault condition exists can be based on a created feedback file. In this case, the feedback file can have a structure which is based on the complexity of the predicted fault condition and/or on the capability profile of the receiver 120a-c. The structure of the feedback file can hereby specify a level of detail of the feedback file. Thus, a feedback file for a fault condition of high complexity can have a structure of higher level of detail than a feedback file for a fault condition of low complexity. Likewise, a feedback file which is transmitted, for example, to the service technician 120b can have a structure of higher level of detail than a feedback file which is transmitted, for example, to the nurse 120a. In this case, the structure of the feedback file can contain, for example, one or more binary questions regarding the predicted fault condition (e.g., “bibag connector correctly attached? Yes/No”). The number of binary questions can be dependent on the complexity of the fault condition.

The structure of the feedback file can likewise contain one or more predefined condition descriptions of the medical device (e.g., “gear pump worn”, “failure of a compressor”). By a combination of one or more of these condition descriptions, a complex situation can also be described. In addition, individual condition descriptions can be highlighted semantically. Should, for example, the service technician 120b consider that mainly a worn gear pump is responsible for a defect of the medical device 110, although the model 130 has only listed the failure of the compressor in the predicted fault condition, he can clarify this via corresponding syntax (e.g., sequence, font, etc.). This way, for example, the main reason can be listed first and only subsequently a supposed secondary reason (“gear pump worn” and “failure of a compressor”). By this optimized type of feedback generation, the prediction quality of the model 130 can be improved. For example, the number of “true negatives” or “false positives” can thus be reduced, thereby improving the prediction quality (e.g., with respect to recall and precision) of the model.

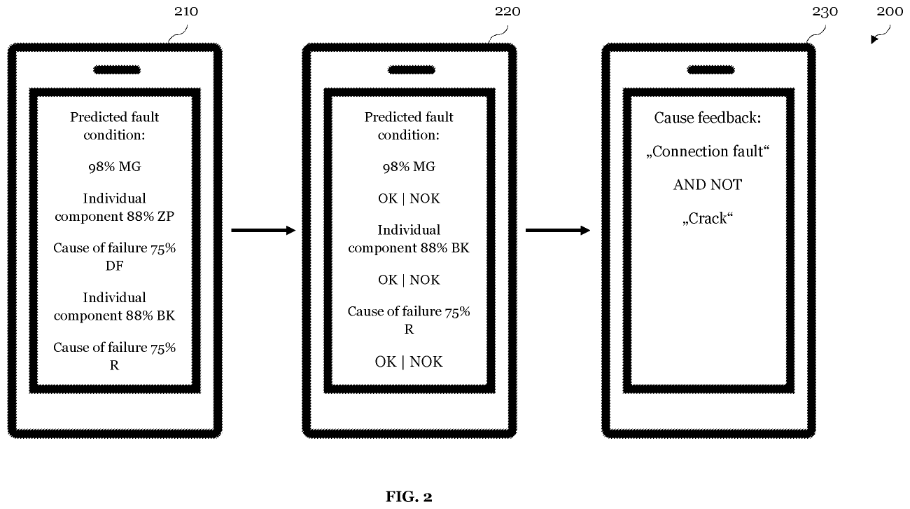

FIG. 2 shows a prediction of a medical device defect including receiver feedback according to aspects of the present disclosure. Any cause that prevents or restricts the treatment of a patient with the medical device 110 can be described as a medical device defect.

Section 210 of FIG. 2 shows a predicted fault condition according to aspects of the present disclosure.

This can comprise a prediction of a probability of failure of the medical device 110 within a predefined future time period (e.g. days, weeks, etc.). For this purpose, the model 130 can comprise a first sub-model, which makes the predictions of the probability of failure within the predefined future time period. In the example shown in section 210, the model 130 or the first sub-model has predicted a probability of failure of the medical device (MG) 110 of 98% within the predefined future time period (e.g. 20 days). It can additionally be predefined from which probability a predicted fault condition is transmitted to a receiver 120a-c. This way, for example, it can be determined (e.g. by a system engineer or one of the receivers 120a-c) that a fault condition is only transmitted from a probability (i.e. probability(s) of failure and/or probability of cause) of 20% or 50%.

In addition or alternatively, the predicted fault condition can comprise one or more predictions of one or more probabilities of failure of one or more individual components of the medical device 110. For this purpose, the model 130 can comprise a first plurality of sub-models, which make the predictions of the one or more probabilities of failure of the one or more individual components of the medical device 100. In this way, a statement can be made about which individual component of the medical device 110 is to be repaired or replaced. The sub-models of the first plurality of sub-models can thus be understood as binary models (e.g. classifiers), wherein a sub-model of the first plurality of sub-models is responsible for the binary prediction of an individual component. In the example shown in section 210, a first sub-model of the first plurality of sub-models of the model 130 has predicted a probability of failure of 88% of the gear pump ZP. A second sub-model of the first plurality of sub-models of the model 130 has additionally predicted a probability of failure of 88% of the bibag connector BK.

In addition or alternatively, the predicted fault condition can comprise one or more predictions of one or more probabilities of cause of failure of the one or more individual components. For this purpose, the model 130 can comprise a second plurality of sub-models, which make the predictions of the one or more probabilities of cause of the failure of the one or more individual components of the medical device 110. The sub-models of the second plurality of sub-models can thus be understood as binary models (e.g. classifiers), wherein a sub-model of the second plurality of sub-models is responsible for the binary prediction of a probability of cause of the failure of the corresponding individual component. In the example shown in section 210, a first sub-model of the second plurality of sub-models of the model 130 has predicted a 75% probability of cause of the failure of the corresponding individual component (in this example the gear pump ZP) on the basis of a disinfecting liquid DF and a second sub-model of the second plurality of sub-models of the model 130 has predicted a 75% probability of cause of the failure of the corresponding individual component (here the BK) on the basis of a crack R.

Section 220 shows an exemplary illustration of a feedback file of the fault condition according to aspects of the present disclosure.

The feedback file can be displayed, for example, on a mobile terminal (e.g. smartphone, tablet, laptop, smartwatch) or a stationary terminal (e.g. desktop personal computer (PC)) or another data processing device of the receiver 120a-c. As described herein, the structure of the feedback file can depend on the complexity of the fault condition and/or on the capability profile of the corresponding receiver 120a-c. In the example shown 220, this is a fault condition which includes a 98% probability of failure of the medical device MG 110. In addition, the fault condition includes an 88% probability of failure of an individual component (here the bibag connector BK) of the medical device 110, which could be the reason for the probability of failure of 98% of the medical device 110. In addition, the fault condition includes a 75% probability of cause of failure of the individual component (here, e.g., a crack R in the bibag connector). The complexity of this fault condition can be determined as low, since it is, for example, visually evaluable. Accordingly, this fault condition could be transmitted to each of the receivers 120a-c. As shown in the example, for this purpose, the structure of the feedback file first has three binary questions, which can be answered either with “OK” (i.e., the prediction is correct) or “NOK” (i.e., the prediction does not apply). If all three questions are answered with “OK”, the predicted fault condition corresponds to the actual operating condition, i.e., there is no difference. In this case, adjusting the set of parameters of the model 130 would not be necessary. However, if one of the predictions does not apply, i.e., there is a difference between the predicted fault condition and the actual operating condition, this information can be communicated to the model 130 and the set of parameters can be adjusted accordingly.

Accordingly, adjusting the set of parameters of the model 130 can also vary. First, according to the actual operating condition or the corresponding information, it can be determined which sub-model(s) of the model 130 is/are to be adjusted. For this purpose, e.g., based on the difference between the predicted fault condition and the actual operating condition, it can be determined in which prediction (e.g., prediction of the probability of failure, prediction of a probability of failure of an individual component or prediction of a probability of cause) the model 130 or the corresponding sub-model was incorrect. Subsequently, a set of parameters of the determined (sub-) model can take place.

For this purpose, section 230 of FIG. 2 shows a corresponding input possibility. If a prediction of the model 130 or of a sub-model is not correct, the receiver 120 a-c can communicate this in the example shown by first answering the corresponding binary question with “NOK”. If the receiver 120 a-c, e.g., checks the bibag connector for a crack and has determined that no crack is recognizable, but the bibag connector is not correctly inserted, the receiver 120 a-c would first answer the predicted probability of cause of failure of the individual component (here the bibag connector) with “NOK”. Subsequently, he can enter the information about the actual operating condition into the feedback file. This way, he can describe the actual operating condition of the medical device 110, e.g., via predefined condition descriptions. In the example mentioned, he could first name the actual cause and then deny the predicted cause via a corresponding logical connection (e.g., “connection fault” AND NOT “crack”). If the model 130 receives this information, a set of parameters of the model 130 can subsequently be adjusted based on the predicted fault condition and the received information about the actual operating condition of the medical device 110. In the example, it could be determined, e.g., that there is a difference between the predicted fault condition and the actual operating condition, namely that the cause of the failure of the bibag connector is a connection fault and not a crack as predicted. Based on this information, it is then possible to determine, on the one hand, the sub-model which is responsible for the cause prediction “connection fault” and, on the other hand, the sub-model which is responsible for the cause prediction “crack”. Subsequently, a set of parameters of the determined sub-models can be adjusted (i.e. re-trained).

The model 130 can thus be understood as a composition (e.g. ensemble) of a plurality of sub-models, wherein each sub-model of the model 130 is responsible (i.e. trained) for a specific prediction. This way, e.g., a first sub-model of the first plurality of sub-models can be configured exclusively for predicting the probability of failure of a specific individual component (e.g. gear pump) of the medical device 110 and a second sub-model of the first plurality of sub-models can be configured exclusively for predicting the probability of failure of another individual component (e.g. bibag connector). Again, a first sub-model of the second plurality of sub-models can be configured exclusively for predicting the probability of cause (e.g. disinfecting fluid) of failure of a specific individual component (e.g. gear pump), a second sub-model of the second plurality of sub-models can be configured exclusively for predicting another probability of cause (e.g. gear failure) of the gear pump and a third sub-model of the second plurality of sub-models can be configured exclusively for predicting a specific probability of cause (e.g. crack) of failure of another individual component (e.g. bibag connector).

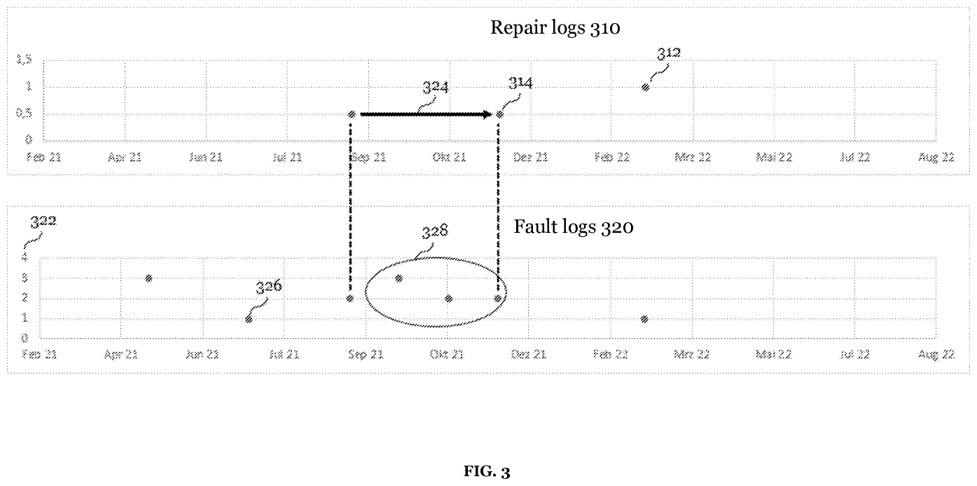

FIG. 3 shows a section 300 of exemplary training data for training a model 130 for predicting medical device defects according to aspects of the present disclosure. The training can hereby also comprise pre-training the model 130 based on the training data.

For this purpose, a plurality of training data is used as input for the model 130. The plurality of training data can be divided according to a training-test ratio (e.g. 80% to 20%). A training file of the plurality of training data can hereby have a fault condition of the medical device that has occurred at a past time, a fault log 320 about an operating condition of the medical device before the past time, and a repair log 312 including actions to eliminate the fault condition that has occurred. The model 130 or the corresponding sub-models can be an XGBOOST classifier with, for example, 100 estimators and a maximum depth of 4. Part of the training can comprise a validation via “K-Fold Cross Validation”.

An exemplary illustration of repair logs 310 is illustrated in the upper part of FIG. 3. One point hereby stands for a created repair log 312 in each case. The highlighted repair log 312 contains, for example, a time of the performed repair (in this example, Feb. 15, 2022) as well as one or more performed actions to eliminate the fault condition of the medical device 110 that has occurred. In this example, the repair log 312 includes, for example, a performed action, wherein a performed action in each case includes a corresponding action code, an affected individual component of the medical device 110 as well as a brief description of the action. In the example shown, the repair log 312 includes a first performed action which includes the action code “1”, the affected individual component “bibag connector” and the description “replacement”.

An exemplary illustration of fault logs 320 is shown in the lower part of FIG. 3. As for the repair logs 310 shown in the upper part of FIG. 3, the X-axis reflects a timeline (here, for example, from February 2021 to August 2022). In the case of the fault logs 320, the Y-axis shows a plurality of fault codes 322 (e.g. fault code 1, fault code 2, etc. to fault code 4). Each fault code of the plurality of fault codes 322 hereby stands for a corresponding fault type or cause. Furthermore, each occurrence of a fault or fault code 326 can be associated with additional information. This information can include a time period (e.g., a number of days) in which the medical device 110 was in use before the fault occurred, a serial number of the medical device 110, and a time of occurrence. In addition, the occurred fault can be labeled (e.g., binary), wherein the label indicates whether a failure of the medical device 110 and/or a failure of an individual component of the medical device 110 has taken place on the basis of the fault. In the case of a binary label, a 1 stands for a failure and a 0 stands for no failure. The labels can either be created manually or approximated. For example, all faults which have occurred within a predefined time, e.g., 20 days, before a failure can be labeled with 1, and faults which have not occurred within the predefined time can be labeled with 0.

These fault logs collected over time can be interpreted as a time series and divided into partial time series 324 via the associated repair logs 310. Thus, a partial time series 324 includes a subset 328 of the fault logs 320 and ends with a corresponding repair log 314. Each of these partial time series can be provided with a corresponding ID. (Counter-based) features can be extracted from each partial time series 324, such as the number of occurrences of a corresponding fault code 322 within the partial time series 324. If a plurality of repair logs 310 have taken place within a short time, the associated partial time series can be combined to form a combined partial time series, since the individual partial time series would otherwise be too short and thus contain too few usable fault logs 320. Thus, a longer partial time series is produced, which contains more usable fault logs 320. The last performed repair log 310 of the plurality of repair logs is then selected as the associated repair log 310. However, it is also possible to combine the repair logs to form a repair log.

An operating condition (i.e. the current operating condition and the operating condition before the past time) can comprise usage data of the medical device 110, technical device data of the medical device 110 and/or environmental data (e.g. temperature of the environment) of the medical device 130. This data can be stored in a corresponding database, which is e.g. part of the system 100, and can be retrieved as required (e.g. for a (pre-training).

Usage data of the medical device 110 can comprise, for example, the device name, the device position (e.g. treatment location or general coordinates such as Global Positioning System (GPS) coordinates), the device manufacturer, the serial number, the device software, the IP address of the medical device 110 within the corresponding network or system 100, a device module configuration (e.g. information about additional modules which have been attached to the medical device 110), a treatment mode of the medical device (e.g. information about a multiplicity of possible treatment modes which the medical device 110 can perform, information about the last used treatment mode, etc.), details about the last treatment (e.g. start and end time of the treatment via the medical device, effective treatment duration such as e.g. the effective dialysis duration), session time (e.g. start and end time), information about warnings during the session (e.g. a list of warnings and/or faults), and/or information about a treatment phase.

Technical device data of the medical device 110 can comprise, for example, a set of parameters of service connection features such as operating duration (e.g. in hours or minutes) of the medical device 110 and/or corresponding modules and/or individual components, hydraulic information (e.g. measured negative pressure Pa at a calibrated rotational speed averaged over a duration of, for example, 5 seconds, measured negative pressure Pa at maximum rotational speed of, for example: 3000 rpm averaged over a duration of, for example, 5 seconds and/or a rotational speed rpm at a predetermined negative pressure of, for example, 0.81-0.85 bar, wherein the rotational speed is measured, for example, by a degassing pump drive) and/or a number of strokes of a dosing pump (e.g. measured via a hardware counter). In addition, the technical device data can contain the information collected in a fault memory of the medical device (e.g. the fault log about an operating condition of the medical device 110 before the past time), sensor parameters of the medical device 110 as well as otherwise detected parameters (e.g. information which is automatically detected in a disinfection routine of the medical device 110). Table 1 shows an exemplary overview of a set of parameters of service connection features, wherein in each case the name of the parameter, a description, the unit, a multiplier as well as a system context (e.g. DB variable, system parameters, etc.) is contained.

Repair logs can contain, for example, information such as time and date of the repair, location of the repair (e.g. clinic), and/or information about the repaired individual components. The actions to eliminate the fault condition that has occurred can include a description of the action. It can thus be detected whether type-identical individual components can be used or can be used in different modules of the medical device (e.g. a pump can be used for the transmission, the ventilation and the flow pump of the medical device). The action to eliminate can also be associated with an action code, whereby actions can be identified.

Method(s) according to the present disclosure can be implemented in the form of a computer program which can be executed on any suitable data processing apparatus comprising correspondingly configured components (e.g. a memory and one or more processors operatively coupled to the memory). The computer program can be stored as computer-executable instructions on a non-transitory computer-readable medium.

Embodiments of the present disclosure can be implemented in various forms. For example, in some embodiments, a computer-implemented method, a computer-readable storage medium or a computer system may be provided.

In some embodiments, a non-transitory computer-readable storage medium can be configured to store program instructions and/or data, which program instructions, when executed by a computer system, cause the computer system to perform a method, e.g. any of the method embodiments described herein or any combination of the method embodiments described herein or any subset of any of the method embodiments described herein or any combination of such subsets.

In some embodiments, a computing device can be configured to include a processor (or set of processors) and a storage medium, the storage medium storing program instructions, the processor configured to read and execute the program instructions from the storage medium, the program instructions executable to implement any of the various method embodiments described herein (or any combination of the method embodiments described herein or any subset of any of the method embodiments described herein or any combination of such subsets). The apparatus can be implemented in a variety of forms.

Although example embodiments have been described above, these embodiments are not intended to limit the scope of the present disclosure, even if only a single embodiment is described with respect to a particular feature. Examples of features mentioned in the disclosure are, unless otherwise specified, to be understood in an illustrative rather than a restrictive sense. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. In particular, with reference to the appended claims, features from dependent claims can be combined with those of the independent claims, and features from the respective independent claims can be combined in any suitable manner and not only in the specific combinations recited in the appended claims.

It will be appreciated that the execution of the various machine-implemented processes and steps described herein may occur via the execution, by one or more respective processors, of processor-executable instructions stored on one or more tangible, non-transitory computer-readable mediums (such as random access memory (RAM), read-only memory (ROM), programmable read-only memory (PROM), and/or another electronic memory mechanism). Thus, for example, operations performed by various components as discussed herein may be carried out according to instructions stored on and/or applications installed on one or more respective computing devices.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for training a model for predicting medical device defects, comprising:
predicting, by a computing system implementing the model, a future fault condition of a medical device based on a current operating condition of the medical device;
determining, by the computing system, a complexity of the predicted fault condition;
transmitting, by the computing system, the predicted fault condition to a receiver based at least in part on the complexity of the predicted fault condition;
receiving, by the computing system, information about an actual operating condition of the medical device; and
adjusting, by the computing system, a set of parameters of the model based at least in part on the information about the actual operating condition and the predicted fault condition of the medical device.

2. The method of claim 1, wherein adjusting the set of parameters of the model comprises:
determining a difference between the actual operating condition and the predicted fault condition;
wherein adjusting the set of parameters is further based on the determined difference.

3. The method of claim 2, wherein determining the difference between the actual operating condition and the predicted fault condition is based on a created feedback file;
wherein a structure of the created feedback file is based at least in part on the complexity of the predicted fault condition and/or on a capability profile of the receiver; and
wherein the structure of the feedback file specifies a level of detail of the feedback file.

4. The method of claim 3, wherein the structure of the feedback file comprises:
one or more binary questions regarding the predicted fault condition of the medical device;
one or more predefined condition descriptions of the medical device; and/or
one or more action instructions associated with the predicted fault condition.

5. The method of claim 1, wherein the future fault condition of the medical device comprises:
a prediction of a probability of failure of the medical device within a predefined future time period;
a prediction of a probability of failure of an individual component of the medical device; and/or
a prediction of a probability of a cause of failure of an individual component of the medical device.

6. The method of claim 5, wherein predicting the future fault condition of the medical device comprises:
making, by a sub-model of the model, the prediction of the probability of failure of the medical device within the predefined future time period;
making, by a sub-model of the model, the prediction of the probability of failure of the individual component of the medical device; and/or
making, by a sub-model of the model, the prediction of the probability of the cause of the failure of the individual component of the medical device.

7. The method of claim 6, wherein adjusting the set of parameters of the model comprises:
determining, according to the actual operating condition, at least one sub-model to be adjusted; and
adjusting a set of parameters of the determined at least one sub-model.

8. The method of claim 1, wherein the method further comprises:
pre-training the model using training data as input for the model;
wherein a training file of the training data comprises:
a fault condition of the medical device that has occurred at a past time;
a fault log about an operating condition of the medical device before the past time; and
a repair log including actions to eliminate the fault condition that has occurred.

9. The method of claim 1, wherein the transmitting comprises:
determining the capability profile of the receiver; and
matching the determined complexity with the capability profile of the receiver;
wherein the capability profile comprises a capability of the receiver to evaluate a fault condition up to a predefined complexity.

10. The method of claim 1, wherein the current operating condition of the medical device comprises: usage data of the medical device, technical device data of the medical device and/or environmental data of the medical device; and/or
wherein the future fault condition of the medical device comprises: a fault diagnosis, a fault probability and/or a time period.

11. A non-transitory computer-readable medium having processor-executable instructions stored thereon for training a model for predicting medical device defects, wherein the processor-executable instructions, when executed, facilitate performance of the following:
predicting, by a computing system implementing the model, a future fault condition of a medical device based on a current operating condition of the medical device;
determining, by the computing system, a complexity of the predicted fault condition;
transmitting, by the computing system, the predicted fault condition to a receiver based at least in part on the complexity of the predicted fault condition;
receiving, by the computing system, information about an actual operating condition of the medical device; and
adjusting, by the computing system, a set of parameters of the model based at least in part on the information about the actual operating condition and the predicted fault condition of the medical device.

12. The non-transitory computer-readable medium of claim 11, wherein adjusting the set of parameters of the model comprises:
determining a difference between the actual operating condition and the predicted fault condition;
wherein adjusting the set of parameters is further based on the determined difference.

13. The non-transitory computer-readable medium of claim 11, wherein the future fault condition of the medical device comprises:
a prediction of a probability of failure of the medical device within a predefined future time period;
a prediction of a probability of failure of an individual component of the medical device; and/or
a prediction of a probability of a cause of failure of an individual component of the medical device.

14. The non-transitory computer-readable medium of claim 11, wherein the processor-executable instructions, when executed, further facilitate performance of the following:
pre-training the model using training data as input for the model;
wherein a training file of the training data comprises:
a fault condition of the medical device that has occurred at a past time;
a fault log about an operating condition of the medical device before the past time; and
a repair log including actions to eliminate the fault condition that has occurred.

15. The non-transitory computer-readable medium of claim 11, wherein the transmitting comprises:
determining the capability profile of the receiver; and
matching the determined complexity with the capability profile of the receiver;
wherein the capability profile comprises a capability of the receiver to evaluate a fault condition up to a predefined complexity.

16. A computing system for training a model for predicting medical device defects, wherein the computing system comprises:
one or more memories having processor-executable instructions stored thereon; and
one or more processors configured to execute the processor-executable instructions to facilitate the following being performed:
predicting, by the model, a future fault condition of a medical device based on a current operating condition of the medical device;
determining a complexity of the predicted fault condition;
transmitting the predicted fault condition to a receiver based at least in part on the complexity of the predicted fault condition;
receiving information about an actual operating condition of the medical device; and
adjusting a set of parameters of the model based at least in part on the information about the actual operating condition and the predicted fault condition of the medical device.

17. The system of claim 16, wherein adjusting the set of parameters of the model comprises:
determining a difference between the actual operating condition and the predicted fault condition;
wherein adjusting the set of parameters is further based on the determined difference.

18. The system of claim 16, wherein the future fault condition of the medical device comprises:
a prediction of a probability of failure of the medical device within a predefined future time period;
a prediction of a probability of failure of an individual component of the medical device; and/or
a prediction of a probability of a cause of failure of an individual component of the medical device.

19. The system of claim 16, wherein the one or more processors are further configured to execute the processor-executable instructions to facilitate the following being performed:
pre-training the model using training data as input for the model;
wherein a training file of the training data comprises:
a fault condition of the medical device that has occurred at a past time;
a fault log about an operating condition of the medical device before the past time; and
a repair log including actions to eliminate the fault condition that has occurred.

20. The system of claim 16, wherein the transmitting comprises:
determining the capability profile of the receiver; and
matching the determined complexity with the capability profile of the receiver;
wherein the capability profile comprises a capability of the receiver to evaluate a fault condition up to a predefined complexity.
